(19) United States
(12) Patent Application Publication
(10) Pub. No.: US 2010/0168414 A1
(43) Pub. Date: Jul. 1, 2010

(54) PROCESSES FOR PREPARING EZETIMIBE AND INTERMEDIATE COMPOUNDS USEFUL FOR THE PREPARATION THEREOF

(75) Inventors: Ana Gavalda Escude, Barcelona (ES); Jordi Bosch i Llado, Barcelona (ES); Ulrike Nettekoven, Allschwil (CH)

Correspondence Address: IP Patent Docketing;K&L GATES LLP, 599 Lexington Avenue, 33rd Floor, New York, NY, 10022-6030, US

(73) Assignee: Medichem S.A., Barcelona (ES)

(21) Appl. No.: 12/295,442

(22) PCT Filed: Sep. 29, 2007
(86) PCT No.: PCT/IB2007/002885

§ 371 Date: Mar. 15, 2010

Related U.S. Application Data
(60) Provisional application No. 60/786,720, filed on Mar. 29, 2006.

(51) Int. Cl. C07D 205/08

(52) U.S. Cl. 540/200

(57) ABSTRACT

The invention relates, in general, to an improved process for converting compounds of Formula II (below) to compounds of Formula III (below), which are key intermediates for the synthesis of ezetimibe, or to ezetimibe itself, wherein in Formulas II and III, R represents hydrogen, alkyl, or a hydroxyl protecting group (e.g., benzyl group, a substituted benzyl group, or a silyl group). The invention further includes the use of the described process and the use of compounds of Formula III made by the described process for the preparation of ezetimibe.

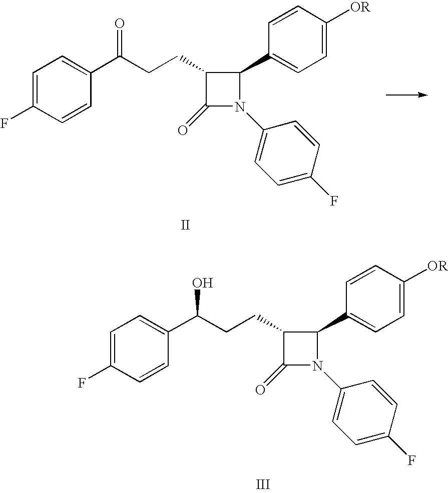

### CROSS REFERENCE TO RELATED APPLICATIONS

This application claims priority to U.S. Provisional Application No. 60/786,720, filed Mar. 29, 2006, which application is expressly incorporated herein by reference in its entirety.

### BACKGROUND OF THE INVENTION

1. Field of the Invention

The invention relates, in general, to an improved process for converting compounds of Formula II (below) to compounds of Formula III (below), which are key intermediates for the synthesis of ezetimibe, or to ezetimibe itself, wherein in Formulas II and III, R represents hydrogen, alkyl, or a hydroxyl protecting group (e.g., benzyl group, a substituted benzyl group, or a silyl group). The invention further includes the use of the described process and the use of compounds of Formula III made by the described process for the preparation of ezetimibe.

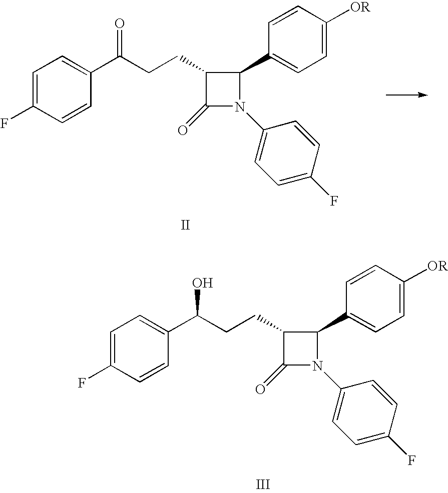

2. Discussion of the Related Art

Ezetimibe is a commercially marketed pharmaceutically active substance known to be useful for the treatment of primary hypercholesterolemia, homozygous familial hypercholesterolemia and homozygous familial sitosterolemia. Ezetimibe has an empirical formula of C₂₄H₂₁F₂NO₃ and a molecular weight of 409.4. Ezetimibe is the international common accepted name for (3R,4S)-1-(4-fluorophenyl)-3-[(3S)-3-(4-fluorophenyl)-3-hydroxypropyl]-4-(4-hydroxyphenyl)azetidin-2-one, and its structural formula is:

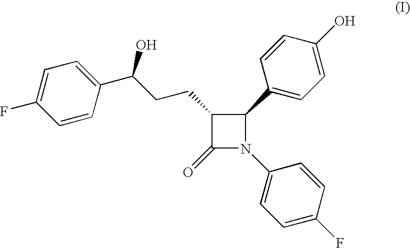

Ezetimibe and its preparation are described in U.S. Reissue Pat. No. 37,721. In this patent, ezetimibe is prepared by the synthetic route shown in Scheme 1 (below):

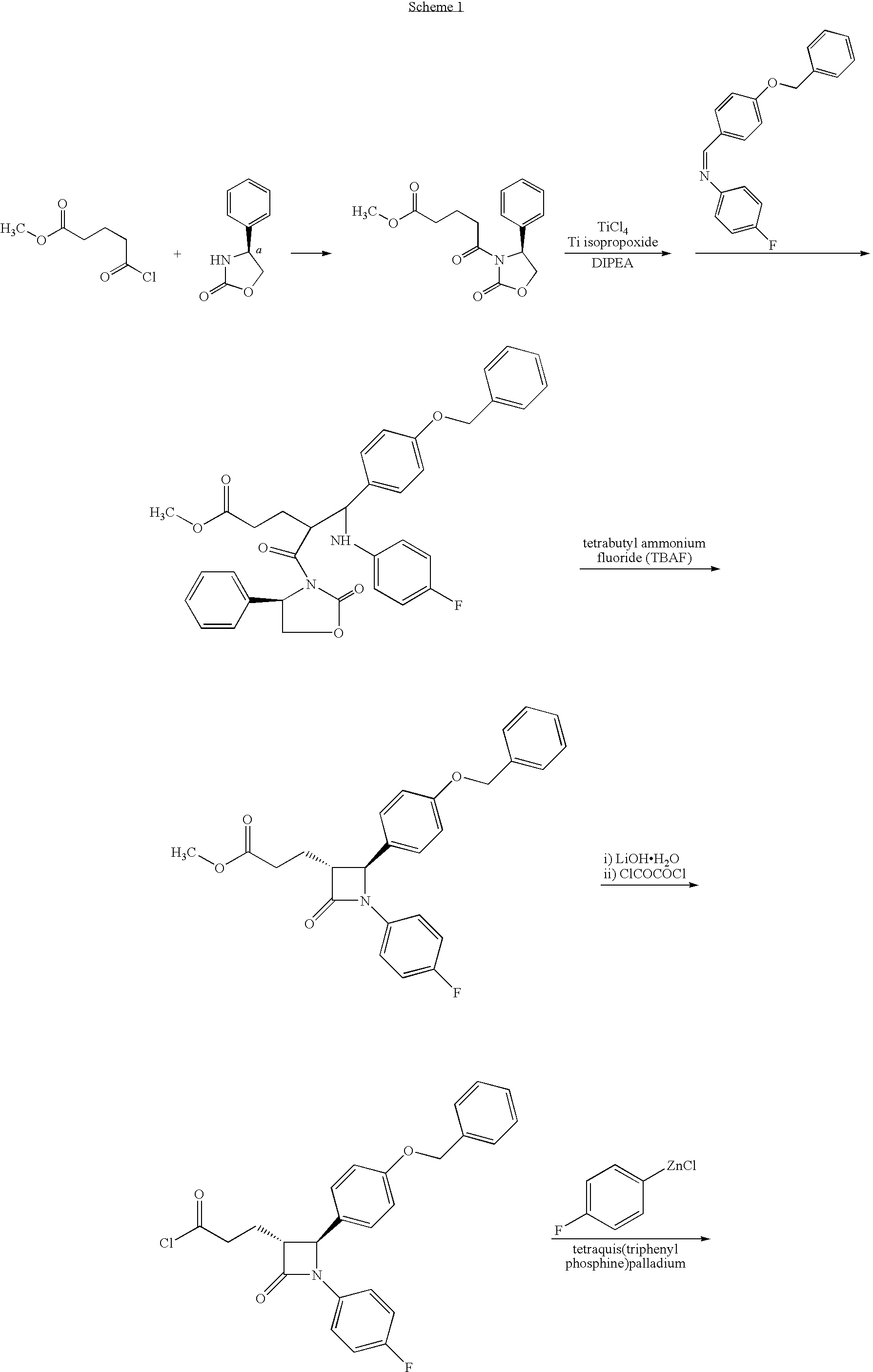

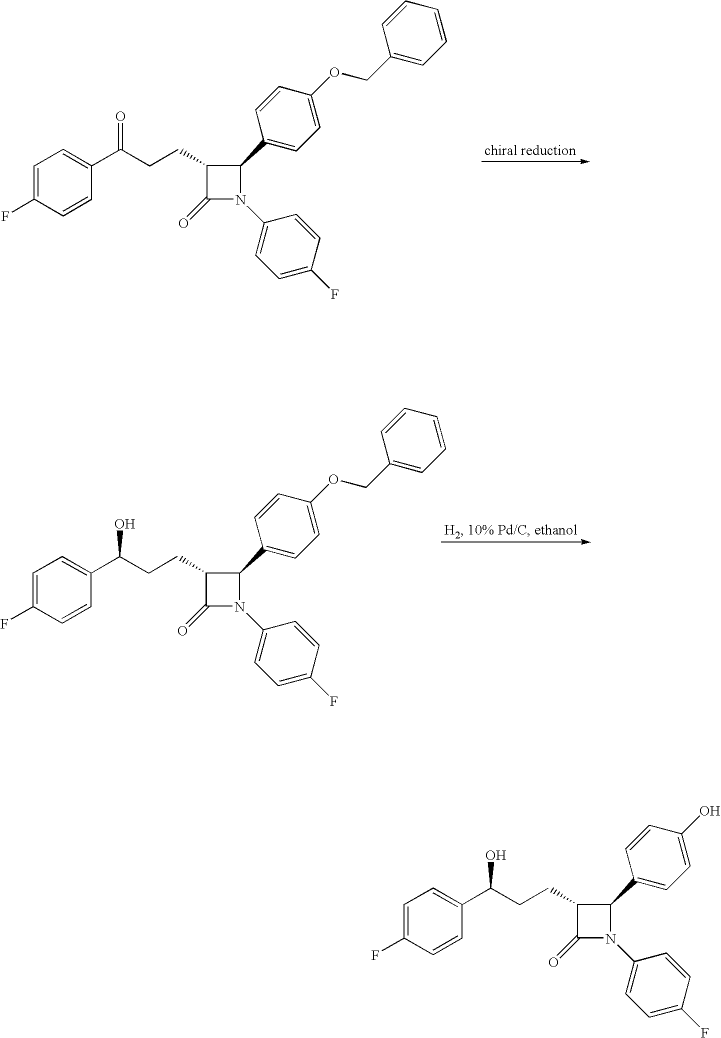

The process described in U.S. Reissue Pat. No. 37,721 and outlined above in Scheme 1 is laborious and involves many steps. As such, there is a need for an improved process for preparing ezetimibe.

Several processes have been described for preparing ezetimibe in, for example, U.S. Pat. Nos. 5,739,321; 5,856,473 and 6,207,822.

U.S. Pat. No. 5,739,321 describes a process for preparing ezetimibe by reacting γ-lactam and an imine to give an azetidinone containing a diol group, which is oxidized to the corresponding aldehyde and then condensed with an enolether. The resulting intermediate is then hydrogenated followed by a chiral catalytic reduction and a debenzylation to yield ezetimibe.

U.S. Pat. No. 5,856,473 describes preparing ezetimibe by oxidation of a propenyl derivative to obtain the corresponding ketone, which is then reduced and debenzylated.

U.S. Pat. No. 6,207,822 describes preparing ezetimibe by reacting p-fluorobenzoylbutyric acid with pivaloyl chloride followed by acylation of the obtained product with a chiral auxiliary. Next, reduction of a keto group is performed using a chiral catalyst. The chiral alcohol thus obtained is then reacted with an imine and a silyl protecting agent to give a 13-(substituted-amino)amide, which is cyclized and then deprotected to yield ezetimibe.

### DETAILED DESCRIPTION OF THE PREFERRED EMBODIMENTS

The invention relates, in general, to an improved process for converting compounds of Formula II (below) to compounds of Formula III (below), which are key intermediates for the synthesis of ezetimibe, or to ezetimibe itself, wherein in Formulas II and III, R represents hydrogen, alkyl, or a hydroxyl protecting group (e.g., a benzyl group, a substituted benzyl group, or a silyl group). The invention further includes the use of the described process and the use of compounds of Formula III made by the described process for the preparation of ezetimibe.

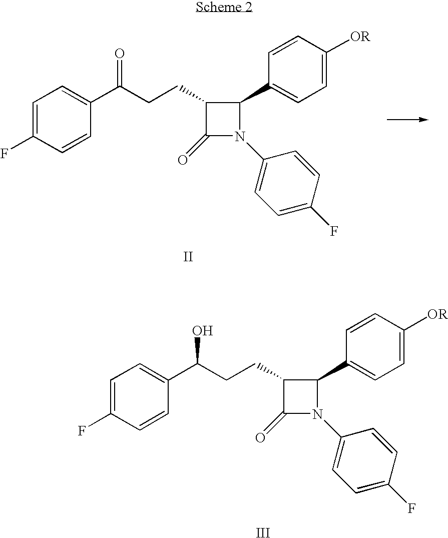

In particular, the invention relates to an improved process for converting compounds of Formula II (e.g., (3R,4S)-4-(4-(benzyloxy)phenyl)-1-(4-fluorophenyl)-3-[3-(4-fluorophenyl)-3-oxopropyl]azetidin-2-one), to compounds of Formula III (e.g., (3R,4S)-4-(4-(benzyloxy)phenyl)-1-(4-fluorophenyl)-3-[(3S)-3-(4-fluorophenyl)-3-hydroxypropyl]azetidin-2-one), which are key intermediates for the synthesis of ezetimibe, wherein in Formulas II and III, R represents hydrogen, alkyl or a hydroxyl protecting group. Preferably R is a benzyl group (see, e.g., Formula IIa, below) or hydrogen (see, e.g., Formula IIb, below). More preferably, R is hydrogen.

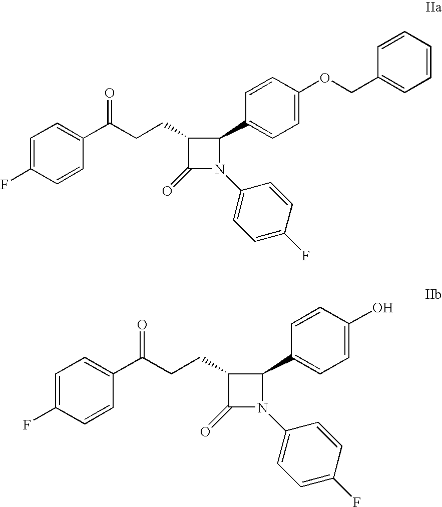

The compounds of Formula II (e.g., (3R,4S)-4-(4-(benzyloxy)phenyl)-1-(4-fluorophenyl)-3-[3-(4-fluorophenyl)-3-oxopropyl]azetidin-2-one) can be converted to compounds of Formula III (e.g., (3R,4S)-4-(4-(benzyloxy)phenyl)-1-(4-fluorophenyl)-3-[(3S)-3-(4-fluorophenyl)-3-hydroxypropyl]azetidin-2-one) via catalytic homogeneous asymmetric reduction of the aryl ketone of the compounds of Formula II.

The catalytic homogeneous asymmetric reduction of the compounds of Formula II to the compounds of Formula III is accomplished using (i) a catalytic homogeneous asymmetric hydrogenation or (ii) a hydrogen transfer-type catalytic homogeneous asymmetric reduction in the presence of either (a) a transition metal complex and an optically active compound or (b) a transition metal complex having an optically active compound as an asymmetric ligand.

Suitable optically active compounds for use on the above-described asymmetric reduction include a nitrogen containing compounds, phosphorus containing compounds and combinations thereof. In particular, the optically active compounds include amino, phosphine, aminophosphine and combinations thereof.

Suitable hydrogen-donating organic or inorganic compounds for use in the hydrogen transfer-type asymmetric reduction include compounds such as 2-propanol, formic acid and formic acid salts.

Suitable transition metals for use in either of the above-described asymmetric reductions include the transition metals from group 8 and group 9.

Suitable transition metals for use in either of the above-described asymmetric reductions include iron, ruthenium, rhodium, iridium and combinations thereof.

Another aspect of the invention includes a process for preparing compounds of Formula III via the above described asymmetric reduction process from the compounds (3R,4S)-4-(4-(benzyloxy)phenyl)-1-(4-fluorophenyl)-3-[3-(4-fluorophenyl)-3-oxopropyl]azetidin-2-one, as depicted in Formula IIa, (3R,4S)-1-(4-fluorophenyl)-3-[3-(4-fluorophenyl)-3-oxopropyl]-4-(4-hydroxyphenyl)-azetidin-2-one, as depicted in Formula IIb and (3R,4S)-4-(4-trimethyl silyloxyphenyl)-1-(4-fluorophenyl)-3-[3-(4-fluorophenyl)-3-oxopropyl]azetidin-2-one.

Another aspect of the invention includes compounds of Formula III prepared from compounds of Formula II by the above-described asymmetric reduction process to prepare ezetimibe.

Another aspect of the invention includes the use of compounds of Formula III prepared from compounds of Formula II by the above-described asymmetric reduction process to prepare ezetimibe.

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

### SPECIFIC EXAMPLES

The following examples are for illustrative purposes only and are not intended, nor should they be interpreted to, limit the scope of the invention.

General Experimental Conditions:

HPLC Chiral Method

The chromatographic separation was carried out in a Daicel CHIRALCEL OD-H, 5 μm, 4.6×150 mm column at room temperature (20-25° C.).

The mobile phase was prepared by mixing 950 mL of hexane with 50 mL of ethanol. The mobile phase was mixed and filtered through 0.22 μm nylon membrane under vacuum.

The chromatograph was equipped with a 232 nm detector and the flow rate was 1 mL per minute. Test samples (10 μl) were prepared by dissolving a sufficient quantity of sample in order to obtain a 0.5 mg per mL concentration in the mobile phase. Following sample injection, the chromatogram was run for at least 60 minutes.

### Preparation of (3R,4S)-4-(4-(benzyloxy)phenyl)-1-(4-fluorophenyl)-3-[(3S)-3-(4-fluorophenyl)-3-hydroxypropyl]azetidin-2-one

As discussed above, the invention relates to an improved process for converting compounds of Formula II to compounds of Formula III, which are key intermediates for the synthesis of ezetimibe and/or ezetimibe itself, wherein in Formulas II and III, R represents hydrogen, alkyl or a hydroxyl protecting group. In the following Examples 1-5, (3R,4S)-4-(4-(benzyloxy)phenyl)-1-(4-fluorophenyl)-3-[(35)-3-(4-fluorophenyl)-3-hydroxypropyl]azetidin-2-one is prepared from (3R,4S)-4-(4-(benzyloxy)phenyl)-1-(4-fluorophenyl)-3-[3-(4-fluorophenyl)-3-oxopropyl]azetidin-2-one via both hydrogen transfer-type catalytic homogeneous asymmetric reduction and catalytic homogeneous asymmetric hydrogenation.

### Example 1

### Hydrogen Transfer-type Reduction

In a 20 mL tube, 250 mg (0.5 mmol) of (3R,4S)-4-(4-(benzyloxy)phenyl)-1-(4-fluorophenyl)-3-[3-(4-fluorophenyl)-3-oxopropyl]azetidin-2-one was dissolved in 2 mL of dimethylformamide. Formic acid (0.08 mL) and triethylamine (0.31 mL) were then added to the mixture with stirring under an argon atmosphere. Next, 16 mg of chloro((S,S)—N-p-toluensulfonyl-1,2-diphenylethylendiamine)(η⁶-p-cymene)ruthenium (obtained from Johnson Matthey Plc) was added, and the mixture was stirred for 48 hours at 30° C. The resulting product was then added to sodium carbonate solution and extracted with dichloromethane. After drying and evaporating the solvent, the obtained product was analyzed by chiral HPLC (Conversion: 94%; d.e.=74%).

### Example 2

### Hydrogen Transfer-type Reduction

In a 20 mL tube, 250 mg (0.5 mmol) of (3R,4S)-4-(4-(benzyloxy)phenyl)-1-(4-fluorophenyl)-3-[3-(4-fluorophenyl)-3-oxopropyl]azetidin-2-one was dissolved in 2.1 mL of a mixture of formic acid (4.4 parts) and triethylamine (2.6 parts). Next, 5.8 mg of chloro((S,S)—N-p-toluensulfonyl-1,2-diphenylethylendiamine)(η⁶-p-cymene)ruthenium (obtained from Johnson Matthey Plc) was added, and the mixture was stirred for 48 hours at 30° C. The product was then added to a sodium carbonate solution and extracted with dichloromethane. After drying and evaporating the solvent, the obtained product was analyzed by chiral HPLC (Conversion: 43%; d.e.=78%).

### Example 3

### Hydrogenation

In a Schlenk flask under an argon atmosphere, 250 mg (0.5 mmol) of (3R,4S)-4-(4-(benzyloxy)phenyl)-1-(4-fluorophenyl)-3-[3-(4-fluorophenyl)-3-oxopropyl]azetidin-2-one was dissolved in 4 mL of freshly distilled toluene. In a separate Schlenk flask under an argon atmosphere, a catalyst (0.005 mmol) of (R)-4-isopropyl-2-[(R)-2-(diphenylphosphino)ferrocen-1-yl]oxazoline triphenylphosphino Ru(II) dichloride was dissolved in 1 mL of toluene. The solutions were then combined under an argon atmosphere via canula in an autoclave. Thereafter, 0.5 mL of a 1M aqueous solution of NaOH that had been purged with argon was added to the mixture in the autoclave via syringe. The autoclave was then purged with argon (three times), with hydrogen (3 times), the pressure was set to 40 bars, and stirring was started. After 18 hours, stirring was stopped, and the pressure was released. Next, 5 mL of water was added to the reaction mixture, and the pH was adjusted to between 5 and 6 by the addition of concentrated acetic acid. The solution was then extracted with dichloromethane. The organic phase was then dried over sodium sulphate, filtered and evaporated to dryness under reduced pressure to yield the product, which was analyzed by HPLC (Conversion: 99%; d.e=94.4).

### Example 4

### Hydrogen Transfer-type Reduction

In a 20 mL tube, 200 mg (0.4 mmol) of (3R,4S)-4-(4-(benzyloxy)phenyl)-1-(4-fluorophenyl)-3-[3-(4-fluorophenyl)-3-oxopropyl]azetidin-2-one was dissolved in 4 mL of toluene. Then, a solution of chloro((S,S)—N-p-toluensulfonyl-1,2-diphenylethylendiamine)(η⁶-p-cymene)ruthenium (obtained from Johnson Matthey Plc) (2.5 mg, 0.004 mmol) in toluene (1.1 mL) was added. The mixture was stirred under nitrogen, triethylamine (0.84 mL, 6 mmol) and formic acid (0.15 mL, 4.0 mmol) were added, and the reaction mixture was stirred for 40 hours at 40° C. The resulting product was then added to sodium hydrogencarbonate solution and washed with water. After evaporating the solvent, the obtained product was analyzed by chiral HPLC (Conversion: 99%; d.e.=85%).

### Example 5

### Hydrogenation

In a 300 mL autoclave, 7.5 g (15 mmol) of (3R,4S)-4-(4-(benzyloxy)phenyl)-1-(4-fluorophenyl)-3-[3-(4-fluorophenyl)-3-oxopropyl]azetidin-2-one was inertized by repeated flushing with 20 bar of argon. In a separate Schlenk flask under an argon atmosphere, a catalyst (0.015 mmol) (R)-4-isopropyl-2-[(R)-2-(diphenylphosphino) ferrocen-1-yl]oxazoline triphenylphosphino Ru(II) dichloride was dissolved in toluene (20 mL). Then, toluene (130 mL) was added to the autoclave via a steel canula, followed by the catalyst solution. A 1 M aqueous solution of NaOH (1 equivalent) that had been purged with argon (3 times) was added to the mixture in the autoclave via syringe. The autoclave was then purged with argon (three times), with hydrogen (3 times), the pressure was set to 40 bars, and stirring was started. After 19 hours, stirring was stopped, and the pressure was released. Next, water (150 mL) was added to the reaction mixture, and the pH was adjusted to between 5 and 6 by the addition of concentrated acetic acid. The solution was then extracted with dichloromethane. The organic phase was then dried over sodium sulfate, filtered and evaporated to dryness under reduced pressure to yield (3R,4S)-4-(4-(benzyloxy)phenyl)-1-(4-fluorophenyl)-3-[(3S)-3-(4-fluorophenyl)-3-hydroxypropyl]azetidin-2-one (7.0 g, 94% yield, conversion: 100%, d.e=95%).

### Preparation of (3R,4S)-1-(4-fluorophenyl)-3-[(3S)-3-(4-fluorophenyl)-3-hydroxypropyl]-4-(4-hydroxyphenyl)-azetidin-2-one (Ezetimibe)

As discussed above, the invention also relates to an improved process for converting compounds of Formula II or Formula III to ezetimibe itself (3R,4S)-1-(4-fluorophenyl)-3-[(3S)-3-(4-fluorophenyl)-3-hydroxypropyl]-4-(4-hydroxyphenyl)-azetidin-2-one; Compound III wherein R is hydrogen). In Example 6 below, ezetimibe is prepared from (3R,4S)-4-(4-(benzyloxy)phenyl)-1-(4-fluorophenyl)-3-[(3S)-3-(4-fluorophenyl)-3-hydroxypropyl]azetidin-2-one. In Example 7 below, ezetimibe is prepared from (3R,4S)-1-(4-fluorophenyl)-3-[3-(4-fluorophenyl)-3-oxopropyl]-4-(4-hydroxyphenyl)-azetidin-2-one via hydrogen transfer-type catalytic homogeneous asymmetric reduction.

### Example 6

### Preparation of Ezetimibe from (3R,4S)-4-(4-(benzyloxy)phenyl)-1-(4-fluorophenyl)-3-[(3S)-3-(4-fluorophenyl)-3-hydroxypropyl]azetidin-2-one

In an inert 100 mL flask, (3R,4S)-4-(4-(benzyloxy)phenyl)-1-(4-fluorophenyl)-3-[(3S)-3-(4-fluorophenyl)-3-hydroxypropyl]azetidin-2-one (0.44 g, 0.88 mmol) and Pd/C (0.1 g) were suspended in ethanol (7.1 mL). The reaction took place under hydrogen atmosphere at room temperature and was followed by TLC. Thereafter, the suspension was filtered over celite, and the solvent was eliminated. The residue was crystallized in MeOH/H₂O to yield (3R,4S)-1-(4-fluorophenyl)-3-[(3S)-3-(4-fluorophenyl)-3-hydroxypropyl]-4-(4-hydroxyphenyl)azetidin-2-one (0.20 g, 0.488 mmol, yield 56%).

### Example 7

### Preparation of Ezetimibe by Hydrogen Transfer-Type Reduction

In a 50 mL Schlenk flask, 500 mg (1.22 mmol) of (3R,4S)-1-(4-fluorophenyl)-3-[3-(4-fluorophenyl)-3-oxopropyl]-4-(4-hydroxyphenyl)-azetidin-2-one were dissolved in 2.0 mL of ethyl acetate. Then, 0.18 mL (1.29 mmol) of triethylamine and 0.05 mL (1.29 mmol) of formic acid were added. The flask was purged with nitrogen, a solution of 7.6 mg (0.012 mmol) of chloro((S,S)—N-p-toluensulfonyl-1,2-diphenylethylendiamine)(η⁶-p-cymene)ruthenium (obtained from Johnson Matthey Plc.) in 1.0 mL of ethyl acetate was added, and the mixture was stirred for 72 hours at 20° C. Then, 0.18 mL (1.29 mmol) of triethylamine and 0.05 mL (1.29 mmol) of formic acid were added, and the reaction was stirred for 24 hours more. The reaction was poured into a sodium hydrogen carbonate solution, extracted with ethyl acetate and washed with water. After drying and evaporating the solvent, 342 mg of a pale yellow solid were obtained (Yield=68%; d.e.=75%).

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

What is claimed is:

1. A process for converting a compound of Formula II to a compound of Formula III 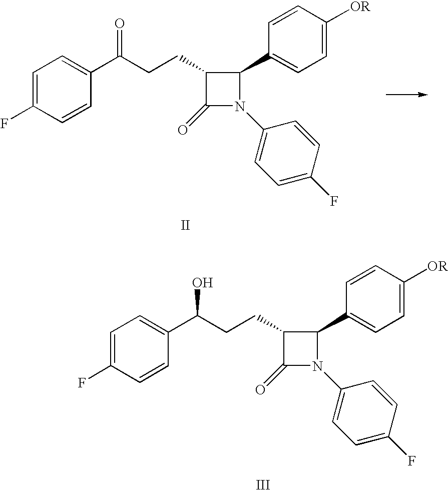
comprising performing a catalytic homogenous asymmetric reduction of the compound of Formula II to produce a compound of Formula III, wherein in Formulas II and III, R represents at least one of hydrogen, a benzyl group and a silyl group; and wherein said catalytic homogenous asymmetric reduction proceeds via at least one of hydrogenation and hydrogen transfer-type reduction.

2. The process of claim 1, wherein said compound of Formula II is at least one of (3R,4S)-4-(4-(benzyloxy)phenyl)-1-(4-fluorophenyl)-3-[3-(4-fluorophenyl)-3-oxopropyl]azetidin-2-one, (3R,4S)-1-(4-fluorophenyl)-3-[3-(4-fluorophenyl)-3-oxopropyl]-4-(4-hydroxyphenyl)-azetidin-2-one and (3R,4S)-4-(4-trimethylsilyloxyphenyl)-1-(4-fluorophenyl)-3-[3-(4-fluorophenyl)-3-oxopropyl]azetidin-2-one.

3. The process of claim 1, wherein said compound of Formula III is at least one of (3R,4S)-4-(4-(benzyloxy)phenyl)-1-(4-fluorophenyl)-3-[(3S)-3-(4-fluorophenyl)-3-hydroxypropyl]azetidin-2-one and (3R,4S)-1-(4-fluorophenyl)-3-[(3S)-3-(4-fluorophenyl)-3-hydroxypropyl]-4-(4-hydroxyphenyl)-azetidin-2-one.

4. The process of claim 1, wherein said catalytic homogenous asymmetric reduction is performed in the presence of at least one transition metal complex having an optically active compound as an asymmetric ligand.

5. The process of claim 4, wherein said at least one transition metal complex is prepared in situ.

6. The process of claim 4, wherein said at least one transition metal complex is prepared prior to said reduction.

7. The process of claim 4, wherein said at least one transition metal complex comprises at least one of iron, ruthenium, rhodium, iridium and combinations thereof.

8. The process of claim 4, wherein said optically active compound is at least one of an amino compound, a phosphine compound, an aminophosphine compound and combinations thereof.

9. The process of claim 1, wherein said catalytic homogeneous asymmetric reduction comprises the use of (R)-4-Isopropyl-2-[(R)-2-(diphenylphosphino)ferrocen-1-yl]oxazoline triphenylphosphino Ru(II) dichloride as a catalyst.

10. The process of claim 1, wherein said catalytic homogeneous asymmetric reduction comprises the use of chloro((S,S)—N-p-toluensulfonyl-1,2-diphenylethylen diamine)(η⁶-p-cymene)ruthenium as a catalyst.

11. The process of claim 1, wherein said catalytic homogeneous asymmetric reduction comprises the use of a hydrogen donating compound.

12. The process of claim 11, wherein said hydrogen donating compound is at least one of 2-propanol, formic acid, formic acid salts and combinations thereof.

13. Ezetimibe prepared according to the process of claim 1.

14. Ezetimibe prepared from a compound of Formula III prepared according to the process of claim 1, wherein R is a benzyl group.
